(19) United States
(12) Patent Application Publication
(10) Pub. No.: US 2019/0224146 A1
(43) Pub. Date: Jul. 25, 2019

(54) COMPOSITIONS AND METHODS OF TREATING AND/OR PREVENTING LYSOSOMAL STORAGE DISEASES AND OTHER MONOGENETIC METABOLIC DISEASES

(71) Applicant: RANEDIS PHARMACEUTICALS, LLC, MEDIA, PA (US)

(72) Inventor: WILLIAM D. CLAYPOOL, MEDIA, PA (US)

(21) Appl. No.: 16/315,760

(22) PCT Filed: Jul. 5, 2017
(86) PCT No.: PCT/US17/40703

§ 371 Date: Jan. 7, 2019

Related U.S. Application Data
(60) Provisional application No. 62/360,012, filed on Jul. 8, 2016.
(60) Provisional application No. 62/513,231, filed on May 31, 2017.

(51) Int. Cl. A61K 31/167; A61K 47/40; A61K 47/10; A61K 47/20

(52) U.S. Cl. CPC A61K 31/167; A61K 47/40; A61P 3/00; A61K 47/20; A61K 47/10

(57) ABSTRACT

The present invention includes compositions for treating a lysosomal storage disease or disorder, and methods using same. In certain embodiments, the compositions of the invention comprise a histone deacetylase inhibitor (HDACi), a cyclodextrin, water, optionally a polyalkylene glycol, and optionally dimethyl sulfoxide (DMSO). In other embodiments, the compositions of the invention comprise a HDACi, a cyclodextrin, water, a polyalkylene glycol, and DMSO.

### CROSS-REFERENCE TO RELATED APPLICATIONS

The present application claims priority under 35 U.S.C. § 119(e) to U.S. Provisional Applications No. 62/513,231, filed May 31, 2017, and No. 62/360,012, filed Jul. 8, 2016, all of which applications are incorporated herein by reference in their entireties.

### BACKGROUND OF THE INVENTION

A lysosome is a membrane-bound organelle that is found in most animal cells and contains hydrolytic enzymes, which can break down virtually all kinds of biomolecules. The lysosome is responsible for polymer degradation, cell secretion, plasma membrane repair, cell signaling and energy metabolism, among other functions. The lysosomes digest unwanted materials in the cytoplasm, both from outside of the cell and from unwanted components inside the cell. Material from the outside of the cell is taken up through endocytosis, while material from the inside of the cell is digested through autophagy. Lysosomes may contain more than 50 different enzymes. These enzymes are synthesized in the rough endoplasmic reticulum and imported from the Golgi apparatus in small vesicles, which fuse with larger acidic vesicles.

Syntheses of lysosomal enzymes are controlled by nuclear genes. Mutations in the genes for these lysosomal enzymes are responsible for more than thirty different human genetic diseases that are collectively known as lysosomal storage diseases (LSDs). These diseases lead to intracellular accumulation of specific substrates that cannot be broken down by the cell.

Monogenic metabolic diseases (MGMDs) are associated with a single mutation in a gene. Certain MGMDs, such as non-ketotic hyperglycinemia, phenylketonuria, and Huntington's Disease, may also lead to organ dysfunction, and particularly, central nervous system dysfunction, because metabolic substrates or products are not handled appropriately as a result of the genetic defect.

LSDs and MGMDs are associated with several neurodegenerative disorders, cancer, cardiovascular diseases, and ageing-related diseases. Non-limiting examples of LSDs comprise Niemann-Pick Disease (NP), Fabry's Disease, Pompe's Disease, Hurler's Disease, Batten's Disease, GM-1 Gangliosidosis, and Gaucher's Disease. LSDs afflict 1:7,500 to 1:10,000 people in the U.S. alone, with approximately 7,000-12,000 LSD patients in the U.S. and a similar number of patients in Europe. In general, treatments for LSDs involve specific enzyme replacement (e.g., administration of β-glucosidase to Gaucher's Disease patients), administration of lipid accumulation inhibitors, and/or palliative treatments.

In the case of NP, lipids accumulate in major organs (e.g., spleen, liver, lungs, bone marrow and/or the brain), causing debilitating conditions such as unsteady gait, abnormal posture, gaze palsy, learning problems, increased sensitivity to touch, spasticity, dementia, seizures, feeding and swallowing difficulties, slurred speech, and enlarged liver and spleen. NP often results in an early death. NP comprise 4 distinct subdiseases: NP Type A (NP-A), NP Type B (NP-B), NP Type C (NP-C) and NP Type D (NP-D). In NP-A and NP-B, insufficient activity of the enzyme sphingomyelinase causes the buildup of toxic amounts of sphingomyelin within the cell. On the other hand, NP-C and NP-D are characterized by defective cellular cholesterol transport, leading to intracellular cholesterol accumulation.

NP-C occurs in an estimated 1:120,000 live births (in the U.S., approximately 33 children per year are born with this disease, and there are fewer than 400 NP-C patients alive at any time). Disease progression can be heterogeneous, but about 50% of cases develop before 10 years of age, and the average life span of NP-C patients following diagnosis is about 6 years. Affected individuals may present enlargement of the spleen and liver, neurodegeneration, loss of vertical and then horizontal saccades (rapid eye movement), seizures, and difficulty in walking, swallowing and speaking. There is no known curative therapy for NP-C, and there is no FDA-standard approved, disease-modifying treatment. Supportive treatment for NP-C is available, but does not modify the presentation or evolution of the disease.

Histone deacetylase inhibitors (HDACis) block activity of HDAC enzymes, thus increasing the degree of acetylation of both histones and non-histone proteins. HDACis affect the transcriptional expression of (mutated) genes, as well as modulate chaperone and proteostatic networks through non-histone proteins. The possibility of using a HDACi for the treatment of LSDs has been raised, but the HDACis' broad activity spectrum raises concerns about undesirable on-target and off-target effects. Further, to be useful in the treatment of LSDs that have a neurodegenerative impact, a HDACi must have good blood brain barrier (BBB) penetration, which is often not the case.

2-Hydroxypropyl-β-cyclodextrin (HPβCD) has also been tested as a possible treatment for LSDs, especially NP-C, due to its ability to chelate cholesterol. However, HPβCD has been found to have poor BBB penetration.

There is thus a need in the art for the identification of compositions that can be used to treat and/or prevent a MGMD and/or a LSD, in a subject. In certain embodiments, the compositions can be used in patients affected with, or at risk of developing a MGMD and/or LSD, such as but not limited to NP-C. The present invention addresses this need.

### BRIEF SUMMARY OF THE INVENTION

The invention provides certain pharmaceutical compositions. The invention further provides methods of treating or preventing a lysosomal storage disease (LSD) and/or a monogenic metabolic disorder (MGMD) in a subject.

In certain embodiments, the composition comprises a histone deacetylase inhibitor (HDACi). In other embodiments, the composition comprises a cyclodextrin. In yet other embodiments, the composition comprises water. In yet other embodiments, the composition comprises a polyalkylene glycol. In yet other embodiments, the composition comprises dimethyl sulfoxide (DMSO). In yet other embodiments, the composition comprises at least one additional agent useful for treating a LSD and/or MGMD.

In certain embodiments, the composition comprises a HDACi, a cyclodextrin, water, optionally a polyalkylene glycol, and optionally DMSO. In other embodiments, the composition comprises a HDACi, a cyclodextrin, water, a polyalkylene glycol, and DMSO. In other embodiments, the relative ratio of polyalkylene glycol, water and DMSO in the composition is about 0-45%:50-100%:0-5%. In yet other embodiments, the % vol/vol of polyethylene glycol in the composition is about 30-60%. In yet other embodiments, the % vol/vol of DMSO in the composition is about 2.5-30%.

In certain embodiments, the composition comprises a polyalkylene glycol. In other embodiments, the composition comprises DMSO. In yet other embodiments, the composition is essentially free of DMSO. In yet other embodiments, the composition is free of DMSO.

In certain embodiments, the relative ratio of polyalkylene glycol, water and DMSO is selected from the group consisting of: about 45%:50%:5%; about 45%:55%:0%; about 40%:60%:0%; about 35%:65%:0%; about 30%:70%:0%; about 25%:75%:0%; about 20%: 80%:0%; about 15%:85%:0%; about 10%:90%:0%; about 5%:95%:0%; and about 0%:100%:0%. In other embodiments, the % vol/vol of polyalkylene glycol (such as but not limited to polyethylene glycol) in the composition is about 35-55%. In yet other embodiments, the % vol/vol of polyethylene glycol (such as but not limited to polyethylene glycol) in the composition is about 30%, 35%, 40%, 45%, 50%, 55% or 60%. In yet other embodiments, the % vol/vol of DMSO in the composition is about 5-25%. In yet other embodiments, the % vol/vol of DMSO in the composition is about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30%.

In certain embodiments, the composition comprises about 5 mg/mL, 4 mg/mL, 3 mg/mL, 2 mg/mL or 1 mg/mL of the HDACi.

In certain embodiments, the composition comprises a cyclodextrin, which concentration is selected from the group consisting of: about 200 mg/mL; about 180 mg/mL; about 160 mg/mL; about 140 mg/mL; about 120 mg/mL; about 100 mg/mL; about 90 mg/mL; about 80 mg/mL; about 70 mg/mL; about 60 mg/mL; about 50 mg/mL; about 40 mg/mL; about 30 mg/mL; about 25 mg/mL; about 20 mg/mL; about 15 mg/mL; about 12.5 mg/mL; about 10 mg/mL; about 8 mg/mL; about 6.5 mg/mL; about 6 mg/mL; about 5 mg/mL; about 4 mg/mL; about 3 mg/mL; about 2.5 mg/mL; about 2 mg/mL; and about 1 mg/mL.

In certain embodiments, the composition allows for blood brain barrier penetration of the HDACi in a subject.

In certain embodiments, the composition is formulated for administration by at least one route selected from the group consisting of nasal, inhalational, rectal, vaginal, pleural, peritoneal, parenteral, topical, transdermal, pulmonary, intranasal, buccal, ophthalmic, epidural, intrathecal, and intravenous. In other embodiments, the composition is formulated for intraperitoneal and/or subcutaneous administration. In yet other embodiments, the subject is administered the composition through at least one route selected from the group consisting of nasal, inhalational, rectal, vaginal, pleural, peritoneal, parenteral, topical, transdermal, pulmonary, intranasal, buccal, ophthalmic, epidural, intrathecal, subcutaneous and intravenous. In yet other embodiments, the subject is administered the composition through at least one route selected from the group consisting of intraperitoneal and subcutaneous.

In certain embodiments, the HDACi is at least one selected from the group consisting of vorinostat, belinostat, LAQ824, panobinostat, givinostat, pyroxamide, trichostatin A, CBHA, and any combinations thereof. In other embodiments, the HDACi is vorinostat. In yet other embodiments, the composition comprises about 5-25 mg/mL of the HDACi. In yet other embodiments, the composition comprises about 10-20 mg/mL of the HDACi. In yet other embodiments, at least a fraction of the HDACi is from a HDACi nanosuspension. In yet other embodiments, the HDACi nanosuspension comprises a dispersant. In yet other embodiments, the dispersant is at least one selected from the group consisting of a polysorbate, poloxamer and (poly)povidone. In yet other embodiments, the composition comprises about 1-3 mg/mL of the dispersant.

In certain embodiments, the cyclodextrin is at least one selected from the group consisting of hydroxypropyl-β-cyclodextrin, 2-hydroxypropyl-β-cyclodextrin (HPβCD), dimethyl-β-cyclodextrin, hydroxypropyl-α-cyclodextrin, hydropropyl-γ-cyclodextrin, sulfobutyl-cyclodextrin, and any combinations thereof. In other embodiments, the cyclodextrin is 2-hydroxypropyl-β-cyclodextrin or sulfobutyl-cyclodextrin. In yet other embodiments, the composition comprises about 200-400 mg/mL of the cyclodextrin.

In certain embodiments, the polyalkylene glycol comprises polyethylene glycol, polypropylene glycol and any mixtures thereof.

In certain embodiments, the method comprises administering to the subject a therapeutically effective amount of a composition of the invention. In other embodiments, the composition is the only therapeutically effective agent administered to the subject. In yet other embodiments, the composition is the only therapeutically effective agent administered to the subject at a therapeutically effective amount to treat and/or prevent a LSD and/or a MGMD.

In certain embodiments, the LSD and/or MGMD comprises at least one selected from the group consisting of Niemann-Pick disease, neuroinflammation due to lysosomal storage disorder, aspartylglucosaminuria, cholesteryl ester storage disease, chronic hexosaminidase A deficiency, cystinosis, Danon disease, Fabry disease, Farber disease, fucosidosis, galactosialidosis, Gaucher's disease, Gaucher disease (types GM1 gangliosidosis, I-cell disease/muco lipidosis II, infantile free sialic acid storage disease/ISSD, juvenile hexosaminidase A deficiency, Krabbe disease, metachromatic leukodystrophy, mucopolysaccharidoses disorders, pseudo-Hurler polydystrophy/mucolipidosis IIIA, MPSI Hurler syndrome, MPSI Scheie syndrome, MPS I Hurler-Scheie syndrome, MPS II Hunter syndrome, Sanfilippo syndrome, Morquio syndrome, MPS IX hyaluronidase deficiency, MPS VI Maroteaux-Lamy, MPS VII Sly syndrome, mucolipidosis I/sialidosis, multiple sulfatase deficiency, neuronal ceroid lipofuscinoses (Batten Disease), Pompe disease, pycnodysostosis, Sandhoff disease, Schindler disease, Salla disease, Tay-Sachs, Huntington's disease, spinal muscular atrophy (SMA), Nonketotic hyperglycinemia, phenylketonuria and Wolman disease. In other embodiments, the LSD comprises Niemann-Pick Disease Type C (NP-C).

In certain embodiments, the subject is further administered at least one additional agent useful for treating a lysosomal storage disease and/or monogenic metabolic disease. In other embodiments, the composition and the at least one additional agent are co-administered to the subject. In yet other embodiments, the composition and the at least one additional agent are coformulated. In yet other embodiments, the subject is a mammal. In yet other embodiments, the subject is a human.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 illustrates sulfobutyl-β-cyclodextrin (R is —(CH₂)₄SO₃Na or H).

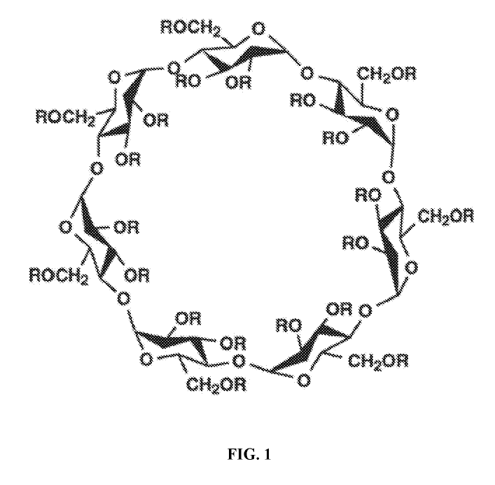

### DETAILED DESCRIPTION OF THE INVENTION

The invention relates, in certain aspects, to the discovery of novel compositions that are effective in treating and/or preventing a LSD (such as but not limited to NP, such as but not limited to NP-C) and/or a MGMD.

The compositions of the invention have improved properties over compositions that are used, or are being developed, for treating and/or preventing LSDs. In certain embodiments, the compositions of the invention are less expensive to prepare and/or develop as therapeutic agents than known compositions in the art. In other embodiments, the compositions of the invention have fewer pharmaceutical development issues than compositions known in the art to treat a LSD and/or a MGMD.

In certain embodiments, the compositions of the invention combine multiple active ingredients and/or excipients, all of which separately and/or in combination increase the tissue availability, plasma exposure and/or tissue exposure of the histone deacetylase inhibitor (such as vorinostat) comprised therein. In other embodiments, the compositions of the invention comprise a cyclodextrin. In yet other embodiments, the compositions of the invention comprise a polyalkylene glycol. In yet other embodiments, the compositions of the invention comprise dimethyl sulfoxide.

### Definitions

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in animal pharmacology, pharmaceutical science, separation science and organic chemistry are those well-known and commonly employed in the art. It should be understood that the order of steps or order for performing certain actions is immaterial, so long as the present teachings remain operable. Moreover, two or more steps or actions can be conducted simultaneously or not.

As used herein, the articles “a” and “an” refer to one or to more than one (i.e., to at least one) of the grammatical object of the article. By way of example, “an element” means one element or more than one element.

As used herein, the term “CD” refers to any cyclodextrin, or derivative thereof.

In one aspect, the terms “co-administered” and “co-administration” as relating to a subject refer to administering to the subject a compound and/or composition of the invention along with a compound and/or composition that may also treat or prevent a disease or disorder contemplated herein. In certain embodiments, the co-administered compounds and/or compositions are administered separately, or in any kind of combination as part of a single therapeutic approach. The co-administered compound and/or composition may be formulated in any kind of combinations as mixtures of solids and liquids under a variety of solid, gel, and liquid formulations, and as a solution (such as a liquid formulation).

The term “container” includes any receptacle for holding the pharmaceutical composition or for managing stability or water uptake. For example, in certain embodiments, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the composition's ability to perform its intended function, e.g., treating, preventing, or reducing a disease or disorder in a patient.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

As used herein, a “dispersant” is a non-surface active polymer or a surface-active substance that is added to a suspension to improve the separation of particles and to prevent settling or clumping. In certain embodiments, dispersants comprise one or more surfactants.

As used herein, the term “DMSO” refers to dimethyl sulfoxide.

As used herein, the term “HDACi” refers to a histone deacetylase inhibitor.

As used herein, the term “HPβCD” refers to 2-hydroxypropyl-β-cyclodextrin.

As used herein, the term “LSD” refers to a lysosomal storage disease.

As used herein, the term “MGMD” refers to a monogenic metabolic disease.

As used herein, the term “NP” refers to a Niemann-Pick Disease. In certain embodiments, NP comprises 4 distinct subdiseases: NP Type A (NP-A), NP Type B (NP-B), NP Type C (NP-C) and NP Type D (NP-D).

As used herein, the term “pharmaceutical composition” or “composition” refers to a mixture of at least one compound useful within the invention with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the invention, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compound prepared from pharmaceutically acceptable non-toxic acids and/or bases, including inorganic acids, inorganic bases, organic acids, inorganic bases, solvates (including hydrates) and clathrates thereof.

As used herein, a “pharmaceutically effective amount,” “therapeutically effective amount” or “effective amount” of a compound is that amount of compound that is sufficient to provide a beneficial effect to the subject to which the compound is administered.

The term “prevent,” “preventing” or “prevention” as used herein means avoiding or delaying the onset of symptoms associated with a disease or condition in a subject that has not developed such symptoms at the time the administering of a compound or composition commences. Disease, condition and disorder are used interchangeably herein.

By the term “specifically bind” or “specifically binds” as used herein is meant that a first molecule preferentially binds to a second molecule (e.g., a particular receptor or enzyme), but does not necessarily bind only to that second molecule.

As used herein, the terms “subject” and “individual” and “patient” can be used interchangeably and may refer to a human or non-human mammal or a bird. Non-human mammals include, for example, livestock and pets, such as ovine, bovine, porcine, canine, feline and murine mammals. In certain embodiments, the subject is human.

The terms “treat,” “treating” and “treatment,” as used herein, means reducing the frequency or severity with which symptoms of a disease or condition are experienced by a subject by virtue of administering an agent or compound to the subject.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual and partial numbers within that range, for example, 1, 2, 2.7, 3, 4, 5, 5.3, and 6. This applies regardless of the breadth of the range.

### Description

The invention relates, in certain aspects, to the discovery of novel compositions that are effective in treating and/or preventing a LSD (such as but not limited to NP, such as but not limited to NP-C), and/or a MGMD (such as but not limited to non-ketotic hyperglycinemia).

In certain embodiments, the compositions of the invention comprise a HDACi, a cyclodextrin and a polyalkylene glycol. In other embodiments, the compositions of the invention allow for the HDACi to have therapeutically effective BBB penetration. In yet other embodiments, the compositions of the invention allow for gene transcriptional changes that affect the onset and/or development of a LSD and/or a MGMD in a subject.

In certain embodiments, the compositions of the invention further comprise water. In other embodiments, the compositions of the invention further comprise saline.

In certain embodiments, the compositions of the invention further comprise dimethyl sulfoxide (DMSO). In other embodiments, the compositions of the invention are essentially free of DMSO. In yet other embodiments, the compositions of the invention are free of DMSO. In yet other embodiments, the compositions of the invention do not cause, or have a manageable presentation of, at least one side effect that may be often associated with administration of DMSO, such as, but not limited to, liver damage, kidney damage, skin reactions, dry skin, headache, dizziness, drowsiness, nausea, vomiting, diarrhea, constipation, breathing problems, vision problems, blood problems, allergic reactions, garlic-like taste, and/or breath and body odor.

In one aspect, the compositions of the invention comprise a HDACi, or a salt, solvate, enantiomer, diastereoisomer, geometric isomer and/or tautomer thereof.

In certain embodiments, the HDACi inhibitor is at least one selected from the group consisting of Class I, Class IIa, Class IIb, and Class IV, and any combinations thereof. In other embodiments, a Class I HDACi inhibits at least one selected from the group consisting of HDAC1, HDAC2, HDAC3, and HDAC8. In yet other embodiments, a Class IIa HDAC inhibitor inhibits at least one selected from the group consisting of HDAC4, HDAC5, HDAC7, and HDAC9. In yet other embodiments, a Class IIb HDAC inhibitor inhibits at least one selected from the group consisting of HDAC6 and HDAC10. In yet other embodiments, a Class IV HDAC inhibitor inhibits at least HDAC11. In yet other embodiments, the HDACi is a hydroxamate or hydroxamic acid. In yet other embodiments, the HDACi is at least one selected from the group consisting of vorinostat (also known as N-hydroxy-N′-phenyloctanediamide or SAHA), belinostat (also known as (2E)-N-Hydroxy-3-[3-(phenylsulfamoyl)phenyl]prop-2-enamide), LAQ824 (also known as (E)-3-(4-(((2-(1H-indol-3-yl)ethyl)(2-hydroxyethyl)amino) methyl)phenyl)-N-hydroxyacrylamide), panobinostat (also known as (2E)-N-hydroxy-3-[4-({[2-(2-methyl-1H-indol-3-yl)ethyl]amino}methyl)phenyl]acrylamide), givinostat (also known as {6-[(diethylamino)methyl]naphthalen-2-yl}methyl [4-(hydroxycarbamoyl)phenyl]carbamate), pyroxamide (also known as N1-Hydroxy-N8-3-pyridinyl-octanediamide), trichostatin A (also known as [R-(E,E)]-7-[4-(Dimethylamino)phenyl]-N-hydroxy-4,6-dimethyl-7-oxo-2,4-heptadienamide), CBHA (m-carboxycinnamic acid bis-hydroxamide), and any combinations thereof. In yet other embodiments, the HDACi comprises vorinostat. In yet other embodiments, the HDACi is vorinostat.

In certain embodiments, the HDACi is used as provided by a commercial source. In other embodiments, at least a fraction of the HDACi used in the compositions of the invention is present in particulate form as part of a suspension. In yet other embodiments, the nanosuspension comprises at least one dispersant. In yet other embodiments, the dispersant is at least one selected from the group consisting of a polysorbate, poloxamer and povidone and any derivatives thereof; cellulosics such as carboxymethyl cellulose or hydroxypropylmethyl cellulose; polyoxyethylene fatty acid esters; pegylated castor oil; polyvinyl alcohol; and bile acid salts. In a non-limiting example, the HDACi is combined with a solution of the dispersant in a carrier fluid, such as but not limited to an aqueous solution, water and/or saline, and the mixture is then milled in order to affect the resulting particle size. The resulting particle size from the milling can target various mean particle sizes from several microns, or alternately to submicron size of 10 to 100's of nanometers. Milling can occur, for example, by agitation in the presence of an inert milling media such as for example grinding beads, for example yttrium-stabilized zirconium oxide, glass, steel, or polymeric grinding beads. Other methods to mill or affect particle size can include high energy mixing, such as those obtained using a rotor-stator mixer, high pressure homogenization, or ultrasonic processing. Alternately, particle size can be reduced in the dry state using methods such as jet-milling or other dry grinding techniques with the resultant particles then dispersed into a carrier fluid. The milled suspension can be used to prepare a composition of the present invention. In certain embodiments, the suspension is prepared under aseptic conditions. In other embodiments, the suspension is sterile filtered. In yet other embodiments, the suspension is sterilized using gamma irradiation. In yet other embodiments, the final concentration of the dispersant in the composition is about 1-20% (w/w %) of the HDACi particulate mass present in the formulation.

Polysorbates are derived from ethoxylated sorbitan (a derivative of sorbitol) esterified with fatty acids. Non-limiting examples include, but are not limited to, polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate), and polysorbate 80 (polyoxyethylene (20) sorbitan monooleate). The number 20 following the term “polyoxyethylene” refers to the total number of oxyethylene —(CH₂CH₂O)— groups in the molecule. The number following the term “polysorbate” relates to the fatty acid associated with the polyoxyethylene sorbitan part of the molecule. Monolaurate is indicated by 20, monopalmitate is indicated by 40, monostearate by 60, and monooleate by 80. Polysorbates are commercially known as Tween, Scattics or Alkest.

Poloxamers are nonionic triblock copolymers composed of a central hydrophobic chain of polyoxypropylene (poly(propylene oxide)) flanked by two hydrophilic chains of polyoxyethylene (poly(ethylene oxide)). Poloxamers are also known by the trade names SYNPERONICS®, PLURONICS® and KOLLIPHOR®. For the generic term “poloxamer”, these copolymers are commonly named with the letter “P” (for poloxamer) followed by three digits, the first two digits×100 give the approximate molecular mass of the polyoxypropylene core, and the last digit×10 gives the percentage polyoxyethylene content (e.g., P407=poloxamer with a polyoxypropylene molecular mass of 4,000 g/mol and a 70% polyoxyethylene content; P188=poloxamer with a polyoxypropylene molecular mass of 1,800 g/mol and a 80% polyoxyethylene content). For the PLURONIC® trade name, coding of these copolymers starts with a letter to define its physical form at room temperature (L=liquid, P=paste, F=flake (solid)) followed by two or three digits. The first digit (two digits in a three-digit number) in the numerical designation, multiplied by 300, indicates the approximate molecular weight of the hydrophobe; and the last digit×10 gives the percentage polyoxyethylene content (e.g., L61=Pluronic with a polyoxypropylene molecular mass of 1,800 g/mol and a 10% polyoxyethylene content). In the example given, poloxamer 181 (P181)=Pluronic L61, poloxamer 407=Pluronic F127. In certain embodiments, the poloxamer comprises poloxamer 188 or poloxamer 407.

Povidones, or polypovidones, are water-soluble polymers made from the monomer N-vinylpyrrolidone (C₆H₉NO)ₙ.

In another aspect, the compositions of the invention comprise a cyclodextrin, or a salt, solvate, enantiomer, diastereoisomer, geometric isomer and/or tautomer thereof.

In certain embodiments, the cyclodextrin is at least one selected from the group consisting of hydroxypropyl-β-cyclodextrin, 2-hydroxypropyl-β-cyclodextrin (HPβCD), dimethyl-3-cyclodextrin, hydroxypropyl-α-cyclodextrin, hydropropyl-γ-cyclodextrin, sulfobutyl-cyclodextrin, and any combinations thereof. In other embodiments, the cyclodextrin comprises β-cyclodextrin. In yet other embodiments, the cyclodextrin comprises HPβCD. In yet other embodiments, the cyclodextrin is HPβCD. In yet other embodiments, the cyclodextrin comprises 2-hydroxypropyl-β-cyclodextrin. In yet other embodiments, the cyclodextrin is 2-hydroxypropyl-β-cyclodextrin. In yet other embodiments, the cyclodextrin comprises sulfobutyl-cyclodextrin. In yet other embodiments, the cyclodextrin is sulfobutyl-cyclodextrin. In yet other embodiments, the cyclodextrin comprises sulfobutyl-β-cyclodextrin. In yet other embodiments, the cyclodextrin is sulfobutyl-β-cyclodextrin.

In certain embodiments, the cyclodextrin has an average molecular weight ranging from about 970 to 6,000 Da. In other embodiments, the cyclodextrin is α-, β-, or γ-cyclodextrin. In yet other embodiments, the cyclodextrin is crosslinked or non-crosslinked. In yet other embodiments, the cyclodextrin is substituted or unsubstituted.

In certain embodiments, the cyclodextrin has an average molecular weight of about 970, about 972, about 980, about 990, about 1,000, about 1,010, about 1,030, about 1,050, about 1,070, about 1,090, about 1,100, about 1,120, about 1,140, about 1,160, about 1,180, about 1,200, about 1,250, about 1,300, about 1,350, about 1,370, about 1,380, about 1,390, about 1,395, about 1,400, about 1,410, about 1,420, about 1,430, about 1,440, about 1,460, about 1,480, about 1,500, about 1,600, about 1,800, about 2,000, about 2,500, about 3,000, about 3,500, about 4,000, about 5,000, about 6,000 Da, or any combinations, fractions and/or multiples thereof. In other embodiments, the cyclodextrin is 2-hydroxypropyl-β-cyclodextrin and has an average molecular weight of about 1,396 Da. In yet other embodiments, the cyclodextrin is α-cyclodextrin and has an average molecular weight of about 973 Da. In yet other embodiments, the cyclodextrin is β-cyclodextrin and has a molecular weight of about 1,135 Da. In yet other embodiments, the cyclodextrin is γ-cyclodextrin and has a molecular weight of about 1,297 Da.

In certain embodiments, the cyclodextrin has an average number of substituents per glucopyranose unit (or degree of substitution) ranging from about 0.5 to 3. In other embodiments, the average number of substituents per glucopyranoses unit is selected from the group consisting of about 0.50, about 0.55, about 0.60, about 0.65, about 0.70, about 0.75, about 0.80, about 0.85, about 0.90, about 0.95, about 1.0, about 1.1, about 1.2, about 1.3, about 1.4, about 1.5, about 1.6, about 1.7, about 1.8, about 1.9, about 2, about 2.2, about 2.4, about 2.5, about 2.6, about 2.7, about 2.8, about 2.9, about 3, and any combinations, fractions and/or multiples thereof.

In certain embodiments, the cyclodextrin is water soluble. In other embodiments, the cyclodextrin has a water solubility at 25° C. that is equal to or higher than about 10 mg/ml. In yet other embodiments, the cyclodextrin has a water solubility at 25° C. selected from the group consisting of about 10, about 20, about 40, about 60, about 100, about 200, about 300, about 400, about 500, about 600 mg/ml, and any combinations, fractions and/or multiples thereof.

In certain embodiments, the cyclodextrin is 2-hydroxypropyl-β-cyclodextrin, having an average molecular weight of about 1,396 Da and an average degree of substitution of about 0.67 hydroxypropyl groups per glucopyranose unit.

In certain embodiments, the average degree of sulfobutyl substitution in a sulfobutyl cyclodextrin is about 4 per 7 glucopyranose units (or about 0.57 sulfobutyl groups per glucopyranoses unit). In other embodiments, the average degree of sulfobutyl substitution in a sulfobutyl cyclodextrin is about 7 per 7 glucopyranose units (or about 1 sulfobutyl group per glucopyranoses unit). In yet other embodiments, the cyclodextrin is sulfobutyl-β-cyclodextrin, having an average degree of substitution of about 0.57 sulfobutyl groups per glucopyranose unit. In yet other embodiments, the cyclodextrin is sulfobutyl-β-cyclodextrin, having an average degree of substitution of about 1.0 sulfobutyl group per glucopyranose unit.

In certain embodiments, the cyclodextrin is replaced at least partially with polyrotaxanes, wherein β-cyclodextrins are threaded along a polymer chain capped with bulky terminal moieties. Non-limiting examples of polyrotaxanes include 2-hydroxypropyl-β-cyclodextrin/plurionic-based polyrotaxanes, biocleavable plurionic/β-cyclodextrin polyrotaxanes, and the like. These and other examples of polyrotaxanes are disclosed in Tamura & Yui, 2014, Scientific Reports 4:4356; and Mondjinou, et al., 2013, Biomacromolecules 14:4189-4197, incorporated herein by reference in their entireties.

In another aspect, the compositions of the invention comprise a polyalkylene glycol, or a salt, solvate, enantiomer, diastereoisomer, geometric isomer and/or tautomer thereof.

In certain embodiments, the polyalkylene glycol comprises polyethylene glycol, polypropylene glycol and any mixtures thereof. In certain embodiments, the polyalkylene glycol comprises polyethylene glycol. In other embodiments, the polyalkylene glycol is polyethylene glycol. In yet other embodiments, the polyalkylene glycol comprises polypropylene glycol. In other embodiments, the polyalkylene glycol is polypropylene glycol.

In certain embodiments, the average molecular weight of the polyalkylene glycol ranges from about 100 to 6,000 Da. In other embodiments, the average molecular weight of the polyalkylene glycol is about 100, about 200, about 300, about 400, about 500, about 600, about 700, about 800, about 900, about 1,000, about 1,200, about 1,400, about 1,600, about 1,800, about 2,000, about 2,100, about 2,200, about 2,300, about 2,400, about 2,500, about 2,600, about 2,700, about 2,800, about 2,900, about 3,000, about 3,500, about 4,000, about 5,000, about 6,000 Da, and any combinations, fractions and/or multiples thereof.

In certain embodiments, the polyethylene glycol has an average molecular weight of about 100-1,000 Da. In other embodiments, the polyethylene glycol has an average molecular weight of about 200-600 Da. In yet other embodiments, the polyethylene glycol has an average molecular weight of about 400 Da.

In certain embodiments, the relative ratio of polyethylene glycol, water and DMSO in the compositions of the invention is about 0-45%:50-100%:0-5%. In other embodiments, the relative ratio of polyethylene glycol, water and DMSO in the compositions of the invention is about 0-45%:50-100%:0-20%. In yet other embodiments, the relative ratio of polyethylene glycol, water and DMSO in the compositions of the invention is about 45%:50%:5%. In yet other embodiments, the relative ratios of polyethylene glycol, water and DMSO in the compositions of the invention is about 45%:55%:0%. In yet other embodiments, the relative ratio of polyethylene glycol, water and DMSO in the compositions of the invention is about 40%:60%:0%. In yet other embodiments, the relative ratio of polyethylene glycol, water and DMSO in the compositions of the invention is about 35%:65%:0%. In yet other embodiments, the relative ratio of polyethylene glycol, water and DMSO in the compositions of the invention is about 30%:70%:0%. In yet other embodiments, the relative ratio of polyethylene glycol, water and DMSO in the compositions of the invention is about 25%:75%:0%. In yet other embodiments, the relative ratio of polyethylene glycol, water and DMSO in the compositions of the invention is about 20%:80%:0%. In yet other embodiments, the relative ratio of polyethylene glycol, water and DMSO in the compositions of the invention is about 15%:85%:0%. In yet other embodiments, the relative ratio of polyethylene glycol, water and DMSO in the compositions of the invention is about 10%:90%:0%. In yet other embodiments, the relative ratio of polyethylene glycol, water and DMSO in the compositions of the invention is about 5%:95%:0%. In yet other embodiments, the relative ratio of polyethylene glycol, water and DMSO in the compositions of the invention is about 0%:100%:0%.

In certain embodiments, the % vol/vol of polyethylene glycol in the compositions of the invention is about 30-60%. In other embodiments, the % vol/vol of polyethylene glycol in the compositions of the invention is about 35-55%. In yet other embodiments, the % vol/vol of polyethylene glycol in the compositions of the invention is about 40-50%. In yet other embodiments, the % vol/vol of polyethylene glycol in the compositions of the invention is about 30%, 35%, 40%, 45%, 50%, 55% or 60%.

In certain embodiments, the % vol/vol of DMSO in the compositions of the invention is about 0-30%, such as for example about 0.5-30%, about 1-30%, about 1.5-30%, about 2-30%, and/or about 2.5-30%. In other embodiments, the % vol/vol of DMSO in the compositions of the invention is about 5-25%. In yet other embodiments, the % vol/vol of DMSO in the compositions of the invention is about 10-20%. In other embodiments, the % vol/vol of DMSO in the compositions of the invention is about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30%.

In certain embodiments, the % vol/vol of polyethylene glycol and DMSO in the compositions of the invention are respectively about 30-60% and about 2.5-30%. In other embodiments, the % vol/vol of polyethylene glycol and DMSO in the compositions of the invention are respectively about 35-55% and about 5-20%. In yet other embodiments, the % vol/vol of polyethylene glycol and DMSO in the compositions of the invention are respectively about 40-50% and about 5-20%.

In certain embodiments, the compositions of the invention comprise about 5-25 mg/mL, or 10-25 mg/mL or 15-25 mg/mL of the HDACi. In other embodiments, the compositions of the invention comprise about 5-20, 10-20 or 15-20 mg/mL of the HDACi. In yet other embodiments, the compositions of the invention comprise about 25 mg/mL of the HDACi. In yet other embodiments, the compositions of the invention comprise about 22.5 mg/mL of the HDACi. In yet other embodiments, the compositions of the invention comprise about 20 mg/mL of the HDACi. In yet other embodiments, the compositions of the invention comprise about 17.5 mg/mL of the HDACi. In yet other embodiments, the compositions of the invention comprise about 15 mg/mL of the HDACi. In yet other embodiments, the compositions of the invention comprise about 12.5 mg/mL of the HDACi. In yet other embodiments, the compositions of the invention comprise about 10 mg/mL of the HDACi. In yet other embodiments, the compositions of the invention comprise about 7.5 mg/mL of the HDACi.

In certain embodiments, the compositions of the invention comprise about 1-5 mg/mL of the HDACi. In other embodiments, the compositions of the invention comprise about 1-20 mg/mL of the HDACi. In yet other embodiments, the compositions of the invention comprise about 5-20 mg/ml. In yet other embodiments, the compositions of the invention comprise about 20 mg/ml, 15 mg/ml, 10 mg/ml or 5 mg/ml. In yet other embodiments, the compositions of the invention comprise about 5 mg/mL of the HDACi. In yet other embodiments, the compositions of the invention comprise about 4 mg/mL of the HDACi. In yet other embodiments, the compositions of the invention comprise about 3 mg/mL of the HDACi. In yet other embodiments, the compositions of the invention comprise about 2 mg/mL of the HDACi. In yet other embodiments, the compositions of the invention comprise about 1 mg/mL of the HDACi.

In certain embodiments, the compositions of the invention comprise about 1-200 mg/mL of the cyclodextrin. In other embodiments, the compositions of the invention comprise about 200-800 mg/mL of the cyclodextrin. In yet other embodiments, the compositions of the invention comprise about 200 mg/ml, 300 mg/ml, 400 mg/ml, 500 mg/ml, 600 mg/ml, 700 mg/ml or 800 mg/ml of the cyclodextrin. In yet other embodiments, the compositions of the invention comprise about 1-400 mg/mL of the cyclodextrin. In yet other embodiments, the compositions of the invention comprise about 200-500 mg/mL of the cyclodextrin. In yet other embodiments, the compositions of the invention comprise about 200-400 mg/mL of the cyclodextrin. In yet other embodiments, the compositions of the invention comprise about 500 mg/mL, 480 mg/mL, 460 mg/mL, 440 mg/mL, 420 mg/mL, 400 mg/mL, 380 mg/mL, 360 mg/mL, 340 mg/mL, 320 mg/mL, 300 mg/mL, 280 mg/mL, 260 mg/mL, 240 mg/mL, 220 mg/mL, 200 mg/mL, 180 mg/mL, 160 mg/mL, 140 mg/mL, 120 mg/mL, 100 mg/mL, 90 mg/mL, 80 mg/mL, 70 mg/mL, 60 mg/mL, 50 mg/mL, 40 mg/mL, 30 mg/mL, 20 mg/mL, 15 mg/mL, 12.5 mg/mL, 10 mg/mL, 9 mg/mL, 8 mg/mL, 7 mg/mL, 6 mg/mL, 5 mg/mL, 4 mg/mL, 3 mg/mL, 2.5 mg/mL, 2 mg/mL, or 1 mg/mL of the cyclodextrin. In yet other embodiments, the compositions of the invention comprise about 400 mg/mL of the cyclodextrin. In yet other embodiments, the compositions of the invention comprise about 300 mg/mL of the cyclodextrin. In yet other embodiments, the compositions of the invention comprise about 200 mg/mL of the cyclodextrin. In yet other embodiments, the cyclodextrin comprises HPβCD. In yet other embodiments, the cyclodextrin comprises sulfobutyl cyclodextrin.

The compounds of the invention may possess one or more stereocenters, and each stereocenter may exist independently in either the (R) or (S) configuration. In certain embodiments, compounds described herein are present in optically active or racemic forms. The compounds described herein encompass racemic, optically active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically active starting materials, chiral synthesis, or chromatographic separation using a chiral stationary phase. A compound illustrated herein by the racemic formula further represents either of the two enantiomers or mixtures thereof, or in the case where two or more chiral center are present, all diastereomers or mixtures thereof.

In certain embodiments, the compounds of the invention exist as tautomers. All tautomers are included within the scope of the compounds recited herein.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

In all of the embodiments provided herein, examples of suitable optional substituents are not intended to limit the scope of the claimed invention. The compounds of the invention may contain any of the substituents, or combinations of substituents, provided herein.

In certain embodiments, the compositions of the invention are formulated for administration through at least one route selected from the group consisting of nasal, inhalational, oral, rectal, vaginal, pleural, peritoneal, parenteral, topical, transdermal, pulmonary, intranasal, buccal, ophthalmic, epidural, intrathecal, subcutaneous, and intravenous. In other embodiments, the compositions of the invention are formulated for intravenous administration. In yet other embodiments, the compositions of the invention are formulated for subcutaneous administration. In yet other embodiments, the compositions of the invention are formulated for parenteral and/or mucosal (such as, for example, rectal) administration.

### Salts

The compounds described herein may form salts with acids or bases, and such salts are included in the present invention. The term “salts” embraces addition salts of free acids or bases that are useful within the methods of the invention. The term “pharmaceutically acceptable salt” refers to salts that possess toxicity profiles within a range that affords utility in pharmaceutical applications. In certain embodiments, the salts are pharmaceutically acceptable salts. Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (or pamoic), methanesulfonic, ethanesulfonic, benzenesulfonic, pantothenic, sulfanilic, 2-hydroxyethanesulfonic, trifluoromethanesulfonic, p-toluenesulfonic, cyclohexylaminosulfonic, stearic, alginic, β-hydroxybutyric, salicylic, galactaric, galacturonic acid, glycerophosphonic acids and saccharin (e.g., saccharinate, saccharate). Salts may be comprised of a fraction of one, one or more than one molar equivalent of acid or base with respect to any compound of the invention.

Suitable pharmaceutically acceptable base addition salts of compounds of the invention include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (or N-methylglucamine) and procaine. All of these salts may be prepared from the corresponding compound by reacting, for example, the appropriate acid or base with the compound.

### Combination Therapies

In one aspect, the compounds of the invention are useful within the methods of the invention in combination with one or more additional agents useful for treating a LSD and/or a MGMD. These additional agents may comprise compounds or compositions identified herein, or compounds (e.g., development-stage and/or commercially available compounds) known to treat, prevent, or reduce the symptoms of a LSD and/or MGMD (e.g., enzyme replacement therapies such as CEREDASE®, or other metabolically active compounds such as miglustat).

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22:27-55). Each equation referred to elsewhere herein may be applied to experimental data to generate a corresponding graph to aid in assessing the effects of the drug combination. The corresponding graphs associated with the equations referred to elsewhere herein are the concentration-effect curve, isobologram curve and combination index curve, respectively.

### Methods

The invention provides a method of treating or preventing a lysosomal storage disease (LSD) and/or a monogenic metabolic disease (MGMD) in a subject.

In certain embodiments, the LSD and/or MGMD comprises at least one selected from the group consisting of Niemann-Pick disease (such as but not limited to Types A-D), neuroinflammation due to lysosomal storage disorder, aspartylglucosaminuria, cholesteryl ester storage disease, chronic hexosaminidase A deficiency, cystinosis, Danon disease, Fabry disease, Farber disease, fucosidosis, galactosialidosis, Gaucher's disease, Gaucher disease (types gangliosidosis (such as GM1 and/or GM2), I-cell disease/muco lipidosis II, infantile free sialic acid storage disease/ISSD, juvenile hexosaminidase A deficiency, Krabbe disease, metachromatic leukodystrophy, mucopolysaccharidoses disorders, pseudo-Hurler polydystrophy/mucolipidosis IIIA, MPSI Hurler syndrome, MPSI Scheie syndrome, MPS I Hurler-Scheie syndrome, MPS II Hunter syndrome, Sanfilippo syndrome, Morquio syndrome, MPS IX hyaluronidase deficiency, MPS VI Maroteaux-Lamy, MPS VII Sly syndrome, mucolipidosis I/sialidosis, multiple sulfatase deficiency, neuronal ceroid lipofuscinoses (Batten Disease), Pompe disease, pycnodysostosis, Sandhoff disease, Schindler disease, Salla disease, Tay-Sachs, nonketotic hyperglycinemia, Huntington's Disease, spinal muscular atrophy (SMA), phenylketonuria, and/or Wolman disease. In other embodiments, the LSD comprises Niemann-Pick Disease (NP). In yet other diseases, the LSD comprises NP Type C (NP-C).

In certain embodiments, the method comprises administering to the subject in need thereof a therapeutically effective amount of at least one composition of the invention. In other embodiments, the composition of the invention is the only therapeutically effective agent administered to the subject. In yet other embodiments, the composition of the invention is the only therapeutically effective agent administered to the subject in an amount sufficient to treat and/or prevent the LSD and/or the MGMD. In yet other embodiments, the at least one composition further comprises at least one additional pharmaceutically acceptable carrier. In yet other embodiments, the subject is further administered at least one additional agent useful for treating the LSD and/or MGMD. In yet other embodiments, the subject is co-administered the at least one composition and the at least one additional agent. In yet other embodiments, the at least one composition and the at least one additional agent are coformulated.

In certain embodiments, the subject is a mammal. In other embodiments, the mammal is a human. In yet other embodiments, the subject is administered the composition through at least one route selected from the group consisting of nasal, inhalational, oral, rectal, vaginal, pleural, peritoneal, parenteral, topical, transdermal, pulmonary, intranasal, buccal, ophthalmic, epidural, intrathecal, subcutaneous and intravenous.

### Pharmaceutical Compositions and Formulations

The invention provides pharmaceutical compositions that are useful to practice certain methods of the invention. Such a pharmaceutical composition is in a form suitable for administration to a subject, and may comprise one or more pharmaceutically acceptable carriers, one or more additional ingredients, or some combination of these.

In certain embodiments, the pharmaceutical compositions useful for practicing the methods of the invention may be administered to deliver a dose of between 1 ng/kg/dose and 100 mg/kg/dose of any biologically active ingredient(s) that may be present in the compositions. In other embodiments, the pharmaceutical compositions useful for practicing the invention may be administered to deliver a dose of between 1 ng/kg/day and 1,000 mg/kg/day of the biologically active ingredient(s).

The relative amounts of the active ingredient(s), the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 99.9% (w/w) active ingredient(s).

Pharmaceutical compositions that are useful in the methods of the invention may be suitably developed for nasal, inhalational, rectal, vaginal, pleural, peritoneal, parenteral, topical, transdermal, pulmonary, intranasal, buccal, ophthalmic, epidural, intrathecal, subcutaneous intravenous or another route of administration. In certain embodiments, the route of administration comprises parenteral and/or mucosal (such as, for example, rectal). A composition useful within the methods of the invention may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient(s), and immunologically-based formulations.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient(s) into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient(s). The amount of the active ingredient(s) is generally equal to the dosage of the active ingredient(s) that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In certain embodiments, the compositions of the invention are formulated using one or more pharmaceutically acceptable excipients or carriers. In certain embodiments, the pharmaceutical compositions of the invention comprise a therapeutically effective amount of at least one compound of the invention and a pharmaceutically acceptable carrier. Pharmaceutically acceptable carriers, which are useful, include, but are not limited to, glycerol, water, saline, ethanol, recombinant human albumin (e.g., RECOMBUMIN®), solubilized gelatins (e.g., GELOFUSINE®), and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or fragrance-conferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the invention may comprise a preservative from about 0.005% to 2.0% by total weight of the composition. The preservative is used to prevent spoilage in the case of exposure to contaminants in the environment. Examples of preservatives useful in accordance with the invention include but are not limited to those selected from the group consisting of benzyl alcohol, sorbic acid, parabens, imidurea and combinations thereof. One such preservative is a combination of about 0.5% to 2.0% benzyl alcohol and 0.05% to 0.5% sorbic acid.

The composition may include an antioxidant and a chelating agent which inhibit the degradation of the compound. Antioxidants for some compounds are BHT, BHA, alpha-tocopherol and ascorbic acid in the exemplary range of about 0.01% to 0.3%, or BHT in the range of 0.03% to 0.1% by weight by total weight of the composition. The chelating agent may be present in an amount of from 0.01% to 0.5% by weight by total weight of the composition. Exemplary chelating agents include edetate salts (e.g., disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient(s) in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient(s) in aqueous or oily solvents may be prepared in substantially the same manner as liquid suspensions, the primary difference being that the active ingredient(s) is/are dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient(s) in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, ionic and non-ionic surfactants, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, such as a mammal, such as a human, may be carried out using known procedures, at dosages and for periods of time effective to treat a disease or disorder contemplated herein. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 0.01 mg/kg to 1000 mg/kg, or about 0.01 mg/kg to 100 mg/kg, of body weight/per day, depending on the tolerability of the HDACi selected. One of ordinary skill in the art would be able to study the relevant factors and make the determination regarding the effective amount of the therapeutic compound without undue experimentation.

The compositions of the invention can be administered as a single dose, or the composition components can be administered separately. For example, in certain non-limiting embodiments, cyclodextrin can be administered separately from the HDACi drug and polyalkylene glycol. In other non-limiting embodiments, the cyclodextrin is administered before or after the HDACi and polyalkylene glycol. In yet other non-limiting embodiments, the cyclodextrin is administered before the HDACi and polyalkylene glycol. In yet other non-limiting embodiments, the HDACi is administered separately from the cyclodextrin and polyethylene glycol. In yet other non-limiting embodiments, the compositions of the invention are administered as single admixtures.

The composition may be administered to a patient as frequently as several times daily, or it may be administered less frequently, such as once a day, once a week, once every two weeks, once a month, or even less frequently, such as once every several months or even once a year or less. It is understood that the amount of composition dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal. Additional dosing regimens such as four days in succession per month may be appropriate in some treatment circumstances.

Actual dosage levels of the active ingredient(s) in the pharmaceutical compositions of this invention may be varied so as to obtain an amount of the active ingredient(s) that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compositions of the invention employed in the pharmaceutical composition at levels lower than that required in order to achieve the desired therapeutic effect and gradually increase the dosage until the desired effect is achieved.

In particular embodiments, one can formulate the composition in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic composition for the treatment of a disease or disorder in a patient.

In certain embodiments, the compositions of the invention are administered to the patient in dosages that range from one to five times per day or more. In other embodiments, the compositions of the invention are administered to the patient in range of dosages that include, but are not limited to, once every day, every two days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any patient will be determined by the attending physician taking all other factors about the patient into account.

Compounds of the invention for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of a compound of the invention is from about 0.5 μg and about 5,000 mg. In some embodiments, a dose of a compound of the invention used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a composition of the invention, alone or in combination with a second pharmaceutical agent; and instructions for using the composition to treat, prevent, or reduce one or more symptoms of a disease or disorder in a patient.

### Administration

Routes of administration of any of the compositions of the invention include inhalational, nasal (such as intranasal), rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, ocular, intradermal, intra-arterial, intravenous, intrabronchial, inhalation, and topical administration.

Suitable compositions and dosage forms include, for example, tablets, capsules, caplets, pills, gel caps, troches, emulsions, dispersions, (nano)suspensions, solutions, syrups, granules, beads, transdermal patches, gels, powders, pellets, magmas, lozenges, creams, pastes, plasters, lotions, discs, suppositories, liquid sprays for nasal administration, dry powder or aerosolized formulations for inhalation, compositions and formulations for intravesical administration and the like. It should be understood that the formulations and compositions that would be useful in the present invention are not limited to the particular formulations and compositions that are described herein.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intrathecal, subcutaneous, intravenous, intraperitoneal, intramuscular, intrasternal injection, and kidney dialytic infusion techniques.

Formulations of a pharmaceutical composition suitable for parenteral administration comprise the active ingredient(s) combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments, the active ingredient(s) is/are provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient(s), additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations include those which comprise the active ingredient(s) in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Rectal Administration

A pharmaceutical composition of the invention may be prepared, packaged, or sold in a formulation suitable for rectal administration. Such a composition may be in the form of, for example, a suppository, a retention enema preparation, and a solution for rectal or colonic irrigation. Suppository formulations may be made by combining the active ingredient(s) with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives. Retention enema preparations or solutions for rectal or colonic irrigation may be made by combining the active ingredient(s) with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

### Additional Administration Forms

Additional dosage forms of this invention include dosage forms as described in U.S. Pat. Nos. 6,340,475, 6,488,962, 6,451,808, 5,972,389, 5,582,837, and 5,007,790. Additional dosage forms of this invention also include dosage forms as described in U.S. Patent Applications Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems:

In certain embodiments, the compositions and/or formulations of the present invention may be, but are not limited to, short-term, rapid-offset, as well as controlled, for example, sustained release, delayed release and pulsatile release formulations, including nano-particulate formulations for nasal administration.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compositions may be formulated with a suitable polymer or hydrophobic material which provides sustained release properties to the compounds. As such, the compounds for use the method of the invention may be administered in the form of microparticles, for example, by injection or in the form of wafers or discs by implantation.

In certain embodiments of the invention, the compounds useful within the invention are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the invention. Accordingly, all values and ranges encompassed by these values and ranges are meant to be encompassed within the scope of the present invention. Moreover, all values that fall within these ranges, as well as the upper or lower limits of a range of values, are also contemplated by the present application. The description of a range should be considered to have specifically disclosed all the possible subranges as well as individual numerical values within that range and, when appropriate, partial integers of the numerical values within ranges. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Example 1

This study sheds light on whether low levels of HPβCD and/or PEG are effective in helping the HDACi cross the blood brain barrier when delivered subcutaneously.

The following non-limiting formulations are used as references for intraperitoneal administration:

Reference Formulation 1 (with DMSO)—in 10 ml (mgs or %):

| vorinostat | 50 mg/10 ml |
|---|---|
| HPβCD | 2,000 mg/10 ml |
| water | 50% |
| PEG | 45% |
| DMSO | 5% |

Reference Formulation 2 (without DMSO)—in 10 ml (mgs or %):

| vorinostat | 50 mg/10 ml |
|---|---|
| HPβCD | 2,000 mg/10 ml |
| water | 55% |
| PEG | 45% |

In one aspect, the need for use of DMSO in the subcutaneous formulation is assessed by comparing brain penetration of vorinostat for the following formulations free of DMSO, as compared to Reference Formulations 1 and 2. Parameters of interest include, but are not limited to, physicochemical changes of solubility and stability, along with ability for the composition to allow for penetration of vorinostat in the blood brain barrier in mice.

### Series 1

Concentration of HPβCD varied (final 10 ml):

| Solution | (1) | (2) | (3) | (4) | (5) | (6) |
|---|---|---|---|---|---|---|
| vorinostat | 50 | 50 | 50 | 50 | 50 | 50 |
| HPβCD | 1,000 | 500 | 250 | 125 | 65 | 25 |
| PEG | 45% | 45% | 45% | 45% | 45% | 45% |

| | |
|---|---|
| water | (balance) |

### Series 2

Concentration of PEG varied (final 10 ml):

| Solution | (7) | (8) | (9) | (10) | (11) | (12) |
|---|---|---|---|---|---|---|
| vorinostat | 50 | 50 | 50 | 50 | 50 | 50 |
| HPβCD | 2,000 | 2,000 | 2,000 | 2,000 | 2,000 | 2,000 |
| PEG | 40% | 35% | 25% | 10% | 5% | 0% |

| | |
|---|---|
| water | (balance) |

### Series 3

Concentration of sulfobutyl cyclodextrin (SBCD) varied (final 10 ml):

| Solution | (13) | (14) | (15) | (16) | (17) | (18) |
|---|---|---|---|---|---|---|
| vorinostat | 50 | 50 | 50 | 50 | 50 | 50 |
| SBCD | 1,000 | 500 | 250 | 125 | 65 | 25 |
| PEG | 45% | 45% | 45% | 45% | 45% | 45% |

| | |
|---|---|
| water | (balance) |

In certain embodiments, 6-8 week old male Balb/c mice (five mice per group) are injected subcutaneously with test solutions and, 30 minutes or 60 minutes after the injection, the animals are sacrificed to measure vorinostat in both blood and various tissue (including brain).

### Example 2

The following are non-limiting procedures to prepare compositions of the invention:

### Procedure A:

The HDACi is dissolved in DMSO. The polyethylene glycol is added to the solution, along with an aqueous solution of the cyclodextrin. Once the solids are dissolved, the formed solution is sterile filtered and sealed in a sterile vial.

### Procedure B:

A fraction of the HDACi to be incorporated in the composition is derived from a dispersant-containing aqueous nanosuspension of the HDACi. To an aqueous solution of the dispersant (for example, about 1% dispersant) is added the HDACi (at a non-limiting concentration of about 5% HDACi). The resulting system is combined with an appropriate inert milling media, and subjected to milling, thus forming a nanosuspension. Analysis of the nanodispersion showed homogeneous dispersion, without any detectable crystalline particulate. Further, the material can be dispensed using a 25G needle.

Separately, a solution of the HDACi in DMSO is combined with the polyethylene glycol and the cyclodextrin (which is optionally provided as an aqueous solution). The resulting solution is combined with an aliquot of the HDACi nanosuspension to provide a solution of known concentration of HDACi. In certain embodiments, using Procedure B allows for preparation of solutions having higher final HDACi concentrations than using Procedure A.

### Example 3

The following solutions were prepared, and found to remain monophasic after preparation (without formation of visible crystalline particulate upon storage).

It should be noted that compositions comprising >800 mg/mL of HPβCD were viscous slurries or wetted solids, and thus not conducive to injection administration to patients.

Compositions comprising >20 mg/mL of vorinostat, prepared according to procedure A (Example 2), proved to be viscous. However, procedure B allowed preparation of compositions comprising as much as 40 mg/mL of the HDACi.

| | | DMSO | | | |
|---|---|---|---|---|---|
| Formu- | Vorinostat | (mL/ | HPβCD | PEG400 | Water |
| lation | (mg/mL) | mL) | (mg/mL) | (mL/mL) | (mL/mL solution) |
| 1 | 10 | 0.1 | 400 | 0.45 | Balance (~0.45) |
| 2 | 10 | 0.2 | 200 | 0.40 | Balance (~0.40) |
| 3 | 10 | 0.15 | 400 | 0.45 | Balance (~0.40) |
| 4 | 15 | 0.15 | 400 | 0.45 | Balance (~0.40) |
| 5 | 10 | 0.20 | 400 | 0.45 | Balance (~0.35) |
| 6 | 15 | 0.20 | 400 | 0.45 | Balance (~0.35) |
| 7 | 10 | 0.15 | 200 | 0.45 | Balance (~0.40) |
| 8 | 10 | 0.20 | 200 | 0.45 | Balance (~0.35) |
| 9 | 5 | 0.05 | 200 | 0.45 | Balance (~0.50) |
| 10 | 20 | 0.05 | 200 | 0.45 | Balance (~0.50) |
| 11 | 20 | 0.05 | 400 | 0.45 | Balance (~0.50) |

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A pharmaceutical composition comprising a histone deacetylase inhibitor (HDACi), a cyclodextrin, water, optionally a polyalkylene glycol, and optionally dimethyl sulfoxide (DMSO), wherein the relative ratio of polyalkylene glycol, water and DMSO is about 0-45%:50-100%:0-5%.

2. The pharmaceutical composition of claim 1, which comprises a polyalkylene glycol.

3. The pharmaceutical composition of claim 1, which (a) comprises DMSO, or (b) is free or essentially free of DMSO.

4. (canceled)

5. (canceled)

6. The pharmaceutical composition of claim 1, wherein the relative ratio of polyalkylene glycol, water and DMSO is selected from the group consisting of: about 45%:50%:5%; about 45%:55%:0%; about 40%:60%:0%; about 35%:65%:0%; about 30%:70%:0%; about 25%:75%:0%; about 20%:80%:0%; about 15%:85%:0%; about 10%:90%:0%; about 5%:95%:0%; and about 0%:100%:0%.

7. The pharmaceutical composition of claim 1, which comprises about 5 mg/mL, 4 mg/mL, 3 mg/mL, 2 mg/mL or 1 mg/mL of the HDACi.

8. The pharmaceutical composition of claim 1, which comprises a cyclodextrin concentration selected from the group consisting of: about 200 mg/mL; about 180 mg/mL; about 160 mg/mL; about 140 mg/mL; about 120 mg/mL; about 100 mg/mL; about 90 mg/mL; about 80 mg/mL; about 70 mg/mL; about 60 mg/mL; about 50 mg/mL; about 40 mg/mL; about 30 mg/mL; about 25 mg/mL; about 20 mg/mL; about 15 mg/mL; about 12.5 mg/mL; about 10 mg/mL; about 8 mg/mL; about 6.5 mg/mL; about 6 mg/mL; about 5 mg/mL; about 4 mg/mL; about 3 mg/mL; about 2.5 mg/mL; about 2 mg/mL; and about 1 mg/mL.

9. A pharmaceutical composition comprising a histone deacetylase inhibitor (HDACi), a cyclodextrin, water, a polyalkylene glycol, and dimethyl sulfoxide (DMSO), wherein the % vol/vol of polyethylene glycol in the composition is about 30-60% and the % vol/vol of DMSO in the composition is about 2.5-30%.

10. The pharmaceutical composition of claim 9, wherein the % vol/vol of polyethylene glycol in the composition is about 35-55%.

11. (canceled)

12. The pharmaceutical composition of claim 9, wherein the % vol/vol of DMSO in the composition is about 5-25%.

13. (canceled)

14. The pharmaceutical composition of claim 9, which comprises about 5-25 mg/mL of the HDACi.

15. (canceled)

16. The pharmaceutical composition of claim 9, wherein at least a fraction of the HDACi is from a HDACi nanosuspension.

17. The pharmaceutical composition of claim 16, wherein the HDACi nanosuspension comprises a dispersant.

18. (canceled)

19. (canceled)

20. The pharmaceutical composition of claim 9, which comprises about 200-400 mg/mL of the cyclodextrin.

21. (canceled)

22. The pharmaceutical composition of claim 1, which is formulated for administration by at least one route selected from the group consisting of nasal, inhalational, rectal, vaginal, pleural, peritoneal, parenteral, topical, transdermal, pulmonary, intranasal, buccal, ophthalmic, epidural, intrathecal, subcutaneous and intravenous.

23. The pharmaceutical composition of claim 1, wherein the HDACi is at least one selected from the group consisting of vorinostat, belinostat, LAQ824, panobinostat, givinostat, pyroxamide, trichostatin A, CBHA, and any combinations thereof.

24. (canceled)

25. The pharmaceutical composition of claim 1, wherein the cyclodextrin is at least one selected from the group consisting of hydroxypropyl-β-cyclodextrin, 2-hydroxypropyl-β-cyclodextrin (HPβCD), dimethyl-β-cyclodextrin, hydroxypropyl-α-cyclodextrin, hydropropyl-γ-cyclodextrin, sulfobutyl-cyclodextrin, and any combinations thereof.

26. (canceled)

27. The pharmaceutical composition of claim 1, wherein the polyalkylene glycol comprises polyethylene glycol, polypropylene glycol, and any mixtures thereof.

28. (canceled)

29. A method of treating or preventing a LSD or a MGMD in a subject, the method comprising administering to the subject a therapeutically effective amount of the composition of claim 1.

30. The method of claim 29, wherein the LSD or MGMD comprises at least one selected from the group consisting of Niemann-Pick disease, neuroinflammation due to lysosomal storage disorder, aspartylglucosaminuria, cholesteryl ester storage disease, chronic hexosaminidase A deficiency, cystinosis, Danon disease, Fabry disease, Farber disease, fucosidosis, galactosialidosis, Gaucher's disease, Gaucher disease (types GM1 gangliosidosis, I-cell disease/muco lipidosis II, infantile free sialic acid storage disease/ISSD, juvenile hexosaminidase A deficiency, Krabbe disease, metachromatic leukodystrophy, mucopolysaccharidoses disorders, pseudo-Hurler polydystrophy/mucolipidosis IIIA, MPSI Hurler syndrome, MPSI Scheie syndrome, MPS I Hurler-Scheie syndrome, MPS II Hunter syndrome, Sanfilippo syndrome, Morquio syndrome, MPS IX hyaluronidase deficiency, MPS VI Maroteaux-Lamy, MPS VII Sly syndrome, mucolipidosis Usialidosis, multiple sulfatase deficiency, neuronal ceroid lipofuscinoses (Batten Disease), Pompe disease, pycnodysostosis, Sandhoff disease, Schindler disease, Salla disease, Tay-Sachs, Huntington's disease, spinal muscular atrophy (SMA), Nonketotic hyperglycinemia, phenylketonuria, and Wolman disease.

31. (canceled)

32. (canceled)

33. (canceled)

34. (canceled)

35. (canceled)

36. (canceled)

37. (canceled)

38. (canceled)

39. The method of claim 29, wherein the subject is administered the composition through at least one route selected from the group consisting of nasal, inhalational, rectal, vaginal, pleural, peritoneal, parenteral, topical, transdermal, pulmonary, intranasal, buccal, ophthalmic, epidural, intrathecal, subcutaneous and intravenous.

40. (canceled)
